(19) United States
(12) Patent Application Publication
(10) Pub. No.: US 2026/0306792 A1
(43) Pub. Date: Oct. 8, 2026

(54) URINARY CATHETER PRODUCTS WITH INCREASED LUBRICITY

(71) Applicant: Hollister Incorporated, Libertyville, IL (US)

(72) Inventors: Satwinder S. Panesar, Foxford (IE); David Pierce, Ballina (IE); David J. Farrell, Ballina (IE)

(21) Appl. No.: 19/482,460

(22) PCT Filed: May 1, 2024
(86) PCT No.: PCT/US2024/027310

§ 371 Date: Nov. 7, 2025

Related U.S. Application Data
(60) Provisional application No. 63/465,019, filed on May 9, 2023.

(51) Int. Cl. A61M 25/00

(52) U.S. Cl. CPC A61M 25/0041; A61M 25/002; A61M 25/0045; A61M 25/007; A61M 2025/0046; A61M 2202/0014; A61M 2202/0496; A61M 2205/0222; A61M 2205/0238; A61M 2209/06; A61M 2210/1089

(57) ABSTRACT

Catheter products that include a catheter (10) with at least one eyelet (E1) with rounded edges (20a,20b,22a,22b) and a hydrophilic coating (24) on the outer surface (18) of the catheter lumen and the rounded edges of the at least one eyelet, a package (150), and a hydration medium (130).

### CROSS-REFERENCE TO RELATED APPLICATION

This application claims the benefit of U.S. Provisional Application Ser. No. 63/465,019, filed May 9, 2023, the disclosure of which is hereby incorporated by reference in its entirety.

### FIELD OF THE DISCLOSURE

The present disclosure relates to urinary catheter products with urinary catheters that include eyelets with rounded edges on the proximal insertion end portion of the catheter tube. More particularly, the present disclosure relates to urinary catheter products with urinary catheters that include a hydrophilic coating on the outer surface and rounded edges of the eyelets of the catheter tube and, even more particularly, catheter products with a residual hydration medium on the inner surface of the catheter tube that hydrates the hydrophilic coating on the rounded eyelets.

### BACKGROUND

Intermittent urinary catheters include a catheter shaft or tube having a proximal insertion end portion that is inserted through the urethra and into the bladder. Once in the bladder, the urine enters the catheter through one or more openings or eyelets. The urine enters into the opening(s) and drains through a drainage lumen of the catheter tube and out of a distal drainage opening in the distal end portion of the catheter shaft.

The surface of a catheter may be coated with a hydrophilic coating that becomes lubricious upon contact with a hydration medium, such as water, to ease insertion of the catheter into the urethra. The lubricious nature of such materials allows the insertion (and subsequent removal) of a catheter to be accomplished with minimum resistance, thereby reducing discomfort and possible injury.

Urinary catheters having lubricious hydrophilic coatings are usually packaged with a hydration or wetting medium such as water or an aqueous solution. They may be packaged so that hydration medium is loose within the package and free to come into contact with the catheter or the fluid only makes contact with the catheter right before insertion into the body cavity.

Although the use of lubricious hydrophilic coatings on catheters with a hydration medium in the packaging is becoming more common, it remains difficult to prepare a lubricious coating which remains lubricious and do not dry out within the package.

Therefore, there is a need for urinary catheters that have a hydrophilic coating and hydration medium included in the packaging, which have an increased lubricity during use.

### SUMMARY

In a first aspect, a urinary catheter product includes a package containing an intermittent urinary catheter and a hydration medium. The intermittent urinary catheter includes a catheter tube having an inner surface defining a lumen for draining urine, the catheter tube having a proximal insertion end portion for advancement through a urethra into a bladder. The intermittent urinary catheter further includes at least one eyelet located in the proximal insertion end portion of the catheter tube and being in communication with the lumen. The at least one eyelet having a transition defined between an outer surface of the catheter tube and a surface of a wall of the catheter tube defining the at least one eyelet. There is a hydrophilic coating on the outer surface of the catheter tube and on the transition of the at least one eyelet. The hydration medium is in the lumen of the catheter at the proximal insertion end portion of the catheter tube and forms a residual coating on the inner surface of the catheter tube, contacting the hydrophilic coating on the transition of the at least one eyelet.

### BRIEF DESCRIPTION OF THE DRAWINGS

FIG. 1 is a perspective view of a prior art urinary catheter;

FIG. 2 is an enlarged cross-sectional view of the proximal insertion end of a urinary catheter of an exemplary embodiment of the current disclosure;

FIG. 3 is an enlarged cross-sectional view of a portion of the catheter wall of the urinary catheter of FIG. 2;

FIG. 4 is a front view of a urinary catheter product of an exemplary embodiment of the current disclosure;

FIG. 5 is a perspective view of a urinary catheter product of an exemplary embodiment of the current disclosure;

FIG. 6 is a front view of a urinary catheter product of an exemplary embodiment of the current disclosure;

FIG. 7 is a cross-sectional view of the proximal insertion end of a urinary catheter of an exemplary embodiment of the current disclosure;

FIG. 8 is a cross-sectional view of the proximal insertion end of a urinary catheter of an exemplary embodiment of the current disclosure; and

FIG. 9 is a graph showing competitive hydration medium viscosity data.

### DETAILED DESCRIPTION OF THE EMBODIMENTS

While the subject matter of the present disclosure is susceptible to embodiments in various forms, there will hereinafter be described presently preferred embodiments with the understanding that the present disclosure is to be considered an exemplification and is not intended to limit the disclosure to the specific embodiments illustrated. The words “a” or “an” are to be taken to include both the singular and the plural. Conversely, any reference to plural items shall, where appropriate, include the singular.

Methods of increasing lubricity can include altering eyelet shape, such as rounding the edge of the eyelets or coating the edges of the eyelets with hydrophilic coating. Additionally, a hydration medium and/or method of hydrating a hydrophilically coated catheter can increase the lubricity upon insertion.

The present disclosure relates to urinary catheter products with intermittent urinary catheters including eyelets that have rounded edges. The urinary catheters include a lubricous hydrophilic coating disposed on the outer surface of the catheter tube or shaft and on the rounded edges of the eyelets. The urinary catheter product further includes a package containing the urinary catheter and a hydration medium which contacts the hydrophilic coating to enhance the lubricity of the urinary catheter so as to ease the insertion of the catheter into the urethra and reduce discomfort during insertion and removal of the catheter.

As a first component of the urinary catheter product, a urinary catheter includes a catheter tube or shaft that has an insertable portion that is inserted through the urethra and into the bladder to drain urine therefrom. The catheter tube includes a proximal end portion which is inserted through into and through the urethra and into the bladder. The proximal end portion may have eyelets or drainage holes that allow urine to drain out of the bladder and into and through the catheter tube. The catheter tube also includes a distal end portion that may have a drainage element, such as a funnel, associated therewith to drain the urine into a collection container, such as a toilet or waste collection bag.

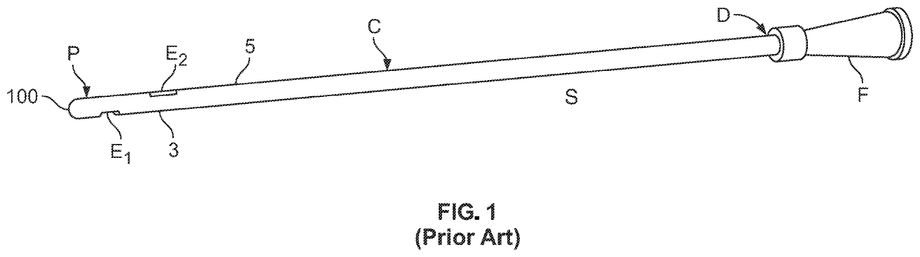

FIG. 1 illustrates a prior art example of an intermittent urinary catheter. Catheter C includes an elongated catheter shaft or tube S having a proximal insertion end portion P and a distal end portion D. Proximal insertion end portion P includes a closed proximal terminal end 100 that is suitable for insertion into the urethra. The proximal insertion end portion includes eyelets or drainage holes E1 and E2 for receiving urine there through and into an internal conduit or lumen of catheter shaft S. Distal end portion D may include a distal opening that is in fluid communication with a drainage member F, such as a funnel, for fluidly connecting catheter C to a collection container, such as a collection bag, or for directing urine to a waste receptacle, such as a toilet.

The catheter includes longitudinal axis A. Although the catheter is an elongated cylinder, there are a number of surfaces that can be defined in relation to the placement of the catheter eyelets. As shown in FIG. 1, there is top surface 3, which is the surface of the first eyelet E1. There is also a second surface, or bottom surface 5, which is the surface of the second eyelet E2.

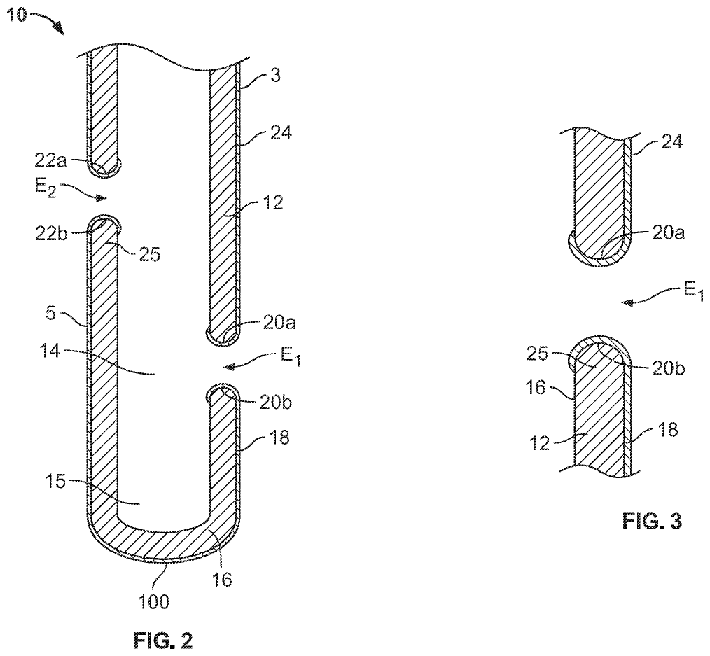

FIGS. 2-3 show enlarged portions of the proximal insert portion of the catheter tube 12 of a urinary catheter 10 which is part of the catheter products of the current disclosure. The unshown portion of the urinary catheter 10 may include any of the components of the prior art catheter of FIG. 1, including the distal end portion. The proximal insertion portion includes eyelets E1 and E2 which open and are in communication with lumen 14 of the catheter tube 12. The lumen 14 is defined by the inner surface 16 of the catheter tube 12. Although two eyelets are shown in certain embodiments, at least one eyelet or more than two eyelets may be present.

The proximal most eyelet (E1 in FIG. 2) may be located between about 2 mm and about 50 mm, or preferably between about 5 mm and about 40 mm from the proximal terminal end 100 of the catheter.

The eyelets of the embodiments disclosed herein may be formed in any suitable manner, including but not limited to, mechanical punching, thermal forming, ultrasonic cutting, laser cutting, molded during a molding process, etc. The drainage holes and eyelets in the embodiments disclosed herein, optionally, are arranged to maximize the drainage ability and efficiency.

In the illustrated embodiment, the eyelets E1, E2 have a generally oval shape. However, in other embodiments, the eyelets may have other shapes, such as oval, circular, irregular, or polygonal. The eyelets may have any of the sizes described above. In alternative embodiments, the eyelets may have various sizes and shapes relative to one another.

The eyelets E1, E2 function to drain urine from the bladder to the inner lumen of the catheter. The eyelets disclosed herein each include an opening or passageway that extends through and is bounded or defined by a surface of catheter wall 25.

As shown in FIGS. 2 and 3, the edges or transitions 20a, 20b, 22a, and 22b of eyelets E1 and E2 between the catheter tube outer surface 18 and the surface of the catheter wall 25 may be rounded transitions or edges. For example, the transitions of the eyelets may be rounded between the outer surface 18 and the catheter wall 25 defining the eyelet. That is, the transition between the outer surface 18 and the catheter wall 25 may have a smooth transition with no angles or virtually no angles. This may be done during the forming of the catheter eyelet or by a later process. In other embodiments, the eyelets transitions between the catheter tube outer surface 18 and the wall 25 may be angled or chamfered.

The catheter 10 with tube 12 includes inside surface 16 and outer surface 18. The outer surface 18 may be coated with hydrophilic coating 24. As shown in FIG. 3, the hydrophilic coating 24 may extend from the outer surface 18 and onto the transitions 20a and 20b of eyelet E1 and transitions 22a and 22b of eyelet E2, and, optionally, extending to contact or cover inner surface 16 defining the catheter lumen 14. The hydrophilic coating 24 may continuously extend over the outer surface, transitions and inner surface.

The hydrophilic coating may include a hydrophilic polymer capable of providing hydrophilicity to the coating and lubriciousness when the coating is hydrated. The polymer may be synthetic or bio-derived and can be blends or copolymers of both. Suitable hydrophilic polymers include but are not limited to poly(lactams), for example polyvinylpyrollidone (PVP), polyurethanes, homo- and copolymers of acrylic and methacrylic acid, polyvinyl alcohol, polyvinylethers, maleic anhydride based copolymers, polyesters, vinylamines, polyethyleneimines, polyethyleneoxides, poly(carboxylic acids), polyamides, polyanhydrides, polyphosphazenes, cellulosics, for example methyl cellulose, carboxymethyl cellulose, hydroxymethyl cellulose, and hydroxypropylcellulose, heparin, dextran, polysacharrides, for example chitosan, hyaluronic acid, alginates, gelatin, and chitin, polyesters, for example polylactides, polyglycolides, and polycaprolactones, polypeptides, for example collagen and fibrins.

In one embodiment, the hydrophilic polymer coating may include poly(lactams), for example polyvinylpyrollidone (PVP), polyurethanes, homo- and copolymers of acrylic and methacrylic acid, polyvinyl alcohol, polyvinylethers, maleic anhydride based copolymers, polyesters, vinylamines, polyethyleneimines or polyethyleneoxides. Preferably, the hydrophilic polymer coating comprises polyvinylpyrrolidone (PVP).

The hydrophilic coating may additionally include various additives such as dispersing and stabilizing agents (surfactants or emulsifiers), antioxidants, photoinitiator and curing agents, such as UV curing agents.

The hydrophilic coating may be applied in any suitable manner. For example, the hydrophilic coating may be applied by dip coating, brushing or spraying. After the coating is applied, the coating is cured by, for example, UV curing.

The catheter product may also include a hydration medium or hydration fluid, indicated as 30, 130, and 416 in the accompanying figures. The hydration medium may be a hydration liquid that includes a liquid, such as water, along with various other components. For example, the hydration medium of the present disclosure includes liquid water and, optionally, one or more of a surface tension reducing agent, mucilage, deep eutectic liquid, oil and osmolality increasing agents. Optionally, the hydration medium may include other additives and agents, such as polyols. Any of the hydration mediums disclosed herein may be used in a foamed or unfoamed state.

Optionally, the hydration medium may be foamed to produce a hydration foam or mousse which may be used to hydrate or wet a device or product. The hydration foam may directly contact the hydrophilic material to hydrate it. The hydration foam includes a mass of gas bubbles formed on or in liquid. Additionally, the creation or formation of the hydration foam may occur at any of the various stages from manufacture to use, depending on the design and use of the medical device. While any of the hydration mediums disclosed herein may be foamed or formed into a foam, depending on the desired use, the hydration mediums disclosed herein also may be employed in an unfoamed state.

Optionally, the hydration medium may include a mucilage. For example, the hydration medium may include water and mucilage, as well as other additives and agents. When a mucilage containing hydration medium is employed, it hydrates the hydrophilic material of the medical device and may form a mucilage layer over the hydrophilic material. For example, when the hydrophilic device is a hydrophilically coated urinary catheter, the hydration medium hydrates the hydrophilic coating and forms a layer containing mucilage over the hydrophilic coating. As will be disclosed in more detail below, the layer of mucilage may have thixotropic properties wherein the layer is thick and viscous under a static condition, and then, becomes less viscous and flows when sheared or otherwise stressed. That is the hydration medium or layer formed therefrom may have a semi-solid or gel-like state and when under stress have more of a liquid state. A hydration medium containing mucilage may be employed in a foamed or unfoamed state.

Optionally, the hydration medium may include a deep eutectic liquid. Deep eutectic liquids can include deep eutectic liquids that are added to the hydration medium and/or and compounds that are added to the hydration medium interact to form a deep eutectic liquid. The hydration medium may include water and a deep eutectic liquid, as well as other additives or agents. Deep eutectic liquids are a mixture or blend of two or more compounds wherein the melting point of the deep eutectic liquid is substantially lower than the melting points of the individual compounds. Deep eutectic liquids have been described as the result of intermolecular hydrogen bonds between the compounds, which at a certain molar ratio leads to a strong depression in the melting point as compared to that of the individual components. Deep eutectic liquids are typically liquid at room temperature (~23° C.). A hydration medium containing a deep eutectic liquid may be employed in a foamed or unfoamed state.

Optionally, the hydration medium may include oil. For example, the hydration medium may include water and oil, as well as other additives and agents. When an oil containing hydration medium is used, it hydrates the hydrophilic material of the medical device and may form a layer containing oil over the hydrophilic material. A hydration medium containing oil may be employed in a foamed or unfoamed state.

In one embodiment, the hydration medium, optionally, includes a viscosifier, such as a combination of high molecular weight polysaccharide and low molecular weight polyols. The high molecular weight polysaccharide may be xanthan gum. The low molecular weight polyols may be glycerol. In some alternatives, the viscosifier includes a high molecular weight polysaccharide at a concentration between 0.1-0.5 wt. % of the hydration medium combined with low molecular weight polyols at a concentration between 1-5 wt. % of the hydration medium. The viscosifier may aid in the formation of mucilage at an interface of hydrated hydrophilic coating. In some alternatives, the hydration medium may optionally include a dispersant. When a dispersant is included, it may be at a concentration between 0.01 wt. % to 2 wt. % of the hydration medium. The dispersant may be anionic surfactant such as sodium methyl cocyl taurate.

The hydration medium may have a particular or desired viscosity. In one embodiment, the hydration medium has a viscosity of from about 4 cP to about 7 cP at 25° C. In a preferred embodiment, the hydration medium has a viscosity from about 4.5 cP to about 6 cP at 25° C. The viscosity may be measured as function of shear rate from 5 to 50 s⁻¹⁻ at 25° C._utilizing the cone and plate method. The hydration medium has a pH from about 3.6 to about 8 and Osmolality of about 40 to about 590 mOsm/dm³. In preferred embodiment pH from about 3.8 to about 7.5 and Osmolality of about 300 to about 550 mOsm/dm³

The urinary catheter product may also include a package or packaging. The package may be any suitable catheter packaging. The package can be single or multilayered and composed of a single piece or multiple pieces. In one embodiment, the packaging includes a sleeve.

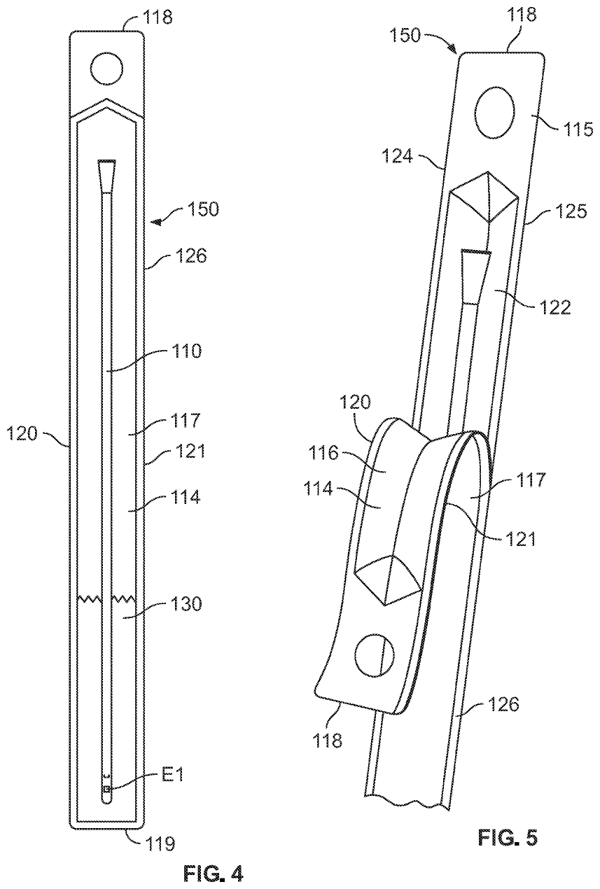

Turning now to FIGS. 4-5, these figures illustrate an embodiment of a urinary catheter product including a package 150 for containing a catheter 110, such as the illustrated catheter, and a hydration medium 130. The package, optionally, could also contain a gripping aid, such as a tubular gripper positioned around the catheter shaft. Optionally, in any of the catheters disclose herein, the distal end of the catheter may be connected to a collection bag. In such embodiment, the collection bag may be contained in the package with the catheter.

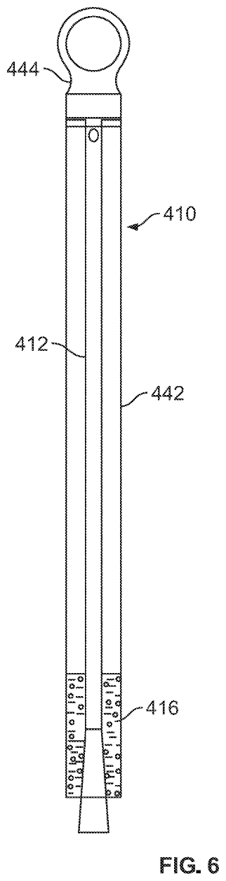

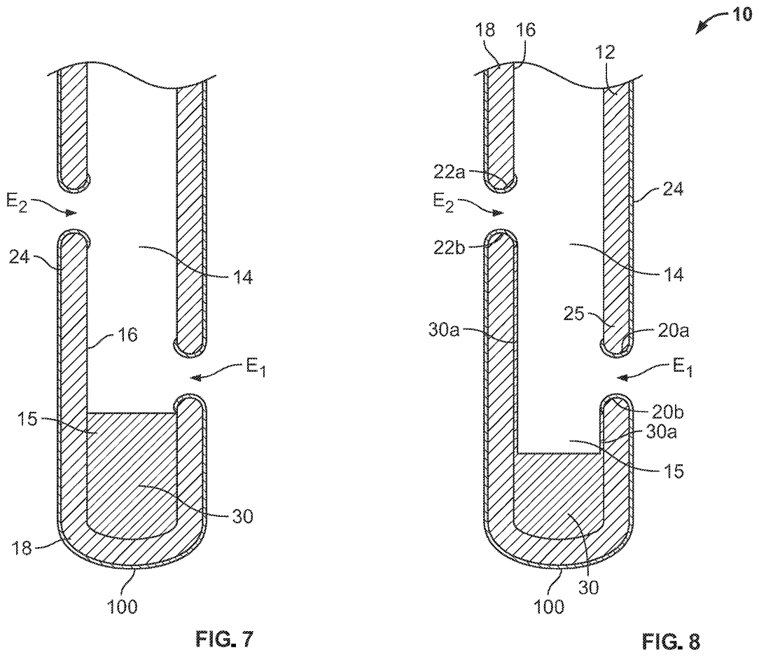

The package 150 may be any suitable package for holding the device. For example, the package may be a tear open package, such as a side tear open package, or a peel open package. In the illustrated embodiment, the package 150 includes a front sheet 114 and a back sheet 115 (FIG. 5). The front sheet 114 includes an inner surface 116 facing the back sheet 115, an outer surface 117 facing the ambient atmosphere, a top edge 118, bottom edge 119 and opposed first and second side edges 120 and 121. Referring to FIGS. 6 and 7, the back sheet 115 includes an inner surface 122 facing the front sheet 114, an outer surface 128 facing the ambient atmosphere, a top edge 123, bottom edge (not shown) and opposed first and second side edges 124 and 125.

The front sheet 114 and back sheet 115 may be made from a liquid and gas impermeable material. For example, the front and back sheets 114 and 115 may be made from a polymer film and/or a metal film. In one embodiment, the material may be a polymer/metal laminate, such as a polymer/aluminum laminate.

The front sheet 114 and back sheet 115 may be sealed to each other to define a cavity for holding the catheter 110. In one embodiment, the front sheet 114 and back sheet 115 may be sealed to each other to form a gas tight cavity. In the illustrated embodiment, the front sheet 114 and back sheet 115 are sealed to each other by a peripheral seal 126. The peripheral seal 126 may be a peelable seal that may be a heat seal, an adhesive seal or any other suitable peelable seal that allows the front sheet 114 to be separated from the back sheet 115 when the sheets are peeled apart during use.

The catheter product 410 also may include a thin flexible sleeve 442 (FIG. 6) that covers at least a section of the outer surface of the catheter 412. The sleeve 442 may be formed of any variety of thin flexible polymeric film materials, such as polyethylene, plasticized PVC, polypropylene, polyurethane, or elastomeric hydrogels. The sleeve 442 may be liquid and/or vapor permeable so as to allow liquid and/or vapor therethrough to hydrate the hydrophilic coating while catheter is stored within the package. Alternatively, the sleeve 442 may include a hydration medium 416 within the sleeve. In embodiments with a sleeve 442, the catheter product may also include a cap 444.

Assembly of the catheter product includes inserting a urinary catheter into a package and adding a hydration medium. The hydration medium can be added any number of conventional ways, resulting a in a sealed product with a urinary catheter and a hydration medium in the package. The hydration medium may be added to the packaging in any known method, such that the hydration medium contacts the hydrophilic coating in the packaging. In cases where a sleeve is utilized, the medium may be added to the sleeve and the catheter and sleeve may then be inserted into a package. In other embodiments, the hydration medium is added directly to the packaging.

FIG. 7 shows the proximal insert portion of the catheter lumen after a hydration medium 30 has been added to the package of the catheter product. As shown in FIG. 7, an amount of hydration medium 30 is retained within the catheter lumen, specifically at the proximal terminal end 100 of the catheter tube 12. For example, a section of the lumen 14 of the catheter tube 12 between the at least one eyelet defines a reservoir 15 containing hydration medium 30. During storage and normal handling (packaging, shipping, etc.), the catheter assembly will be moved and/or jostled during storage and transportation and before use. This movement will cause the hydration medium 30 to move in the packaging, including within the lumen 14 of the catheter tube 12. The hydration medium, when at a selected viscosity, will adhere to the interior surface 16 defining the catheter lumen 14, forming a layer or residual coating 30a on portions of the hydrophilic coating. Additionally, the hydration medium will enter the reservoir 15. Due to the viscosity and flow characteristics of the hydration medium 30, an amount of hydration medium will remain in the reservoir and will resist leaving the reservoir regardless of the orientation of the catheter tube 12 or the package of the catheter product.

FIG. 8 illustrates the resulting urinary catheter 10 proximal insertion end portion after movement of the package, with the hydration medium forming a residual coating 30a on the interior surface 16 defining the catheter lumen 14. Because of the viscosity and composition of the fluid, the coating adheres and contacts the hydrophilic coating. The residual coating 30a maintains the hydration of the hydrophilic coating 24 on the transitions 20a, 20b, 22a, 22b. Optionally, the contact between the residual coating 30a of the hydration medium 30 and the hydrophilic coating 24 continuously hydrates or maintains hydration of the hydrophilic coating. This continuous hydration may occur due to a capillary effect in which the hydrophilic coating 24 draws in hydration medium from the residual coating 30a. This results in an increased lubricity of the urinary catheter, easing insertion when used.

### EXAMPLE

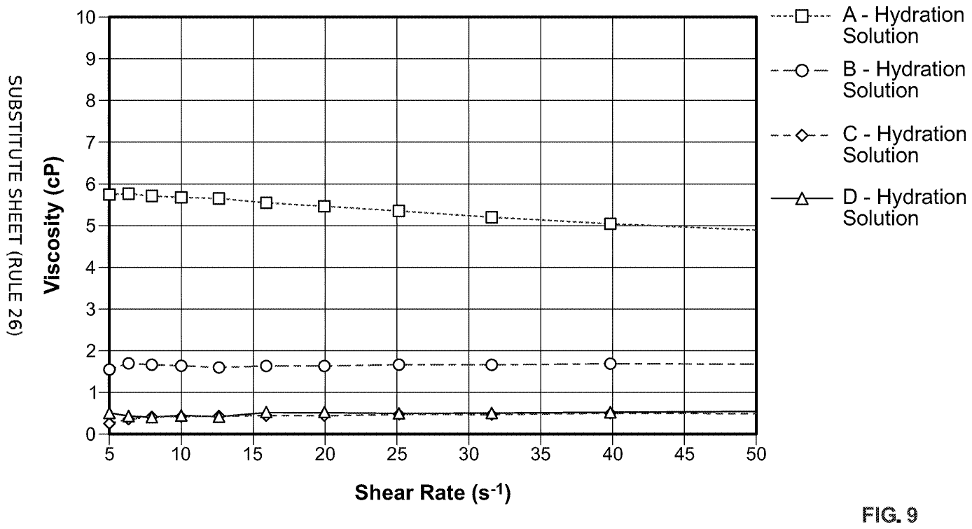

Results from the comparative viscosity testing are shown in FIG. 9., wherein the hydration mediums represented by A-H as shown in Table 1 below. A hydration medium was made by adding 2-4 wt. % of glycerol, 0.1-0.2 wt. % of xanthan gum and 0.2 wt. % of sodium methyl cocyl taurate into de-ionized water. Specifically, xanthan gum was wetted with glycerol to form a paste. The paste was added to water in a water tank and stirred until a homogeneous solution was attained. Then, sodium methyl cocyl taurate was added into the homogenous solution and stirred gently to form a hydration medium identified as “A” in table 1.

The viscosity of the hydration mediums A-H (in table 1 below) were measured by Kinexus Rheometer (Model KNX2100) where viscosity is measured as function of shear rate of 5 to 50 s⁻¹⁻ at 25° C. utilizing the cone and plate method.

TABLE 1

| | |
|---|---|
| A-Hydration Fluid | Hydration Medium |
| B-Hydration Fluid | SpeediCath ® Standard |
| C-Hydration Fluid | Teleflex ® FloCath Quick ™ |
| D-Hydration Fluid | LoFric ® Sense ™ |

The viscosity of the hydration medium A, which may be included in an embodiment of a catheter product of the current disclosure, has a viscosity at 25° C. which, while being numerically low, is measurably higher than other competitive hydration mediums as shown in the graph of FIG. 9. The viscosity is compared to shear rate over time. The viscosity drops as the shear rate increases for hydration medium A, indicating a pseudoplastic behavior, or that the viscosity drops as the shear rate increases.

It should be understood that various changes and modifications to the presently preferred embodiments described herein will be apparent to those skilled in the art. Such changes and modifications can be made without departing from the spirit and scope of the invention disclosed herein.

What is claimed is:

1. A urinary catheter product comprising:
a package containing:
an intermittent urinary catheter comprising:
a catheter tube having an inner surface defining a lumen for draining urine, the catheter tube having a proximal insertion end portion for advancement through a urethra into a bladder;
at least one eyelet located in the proximal insertion end portion of the catheter tube and being in communication with the lumen, the at least one eyelet having a transition defined between an outer surface of the catheter tube and a surface of a wall of the catheter tube defining the at least one eyelet; and
a hydrophilic coating on the outer surface of the catheter tube and on the transition of the at least one eyelet; and
a hydration medium in the lumen of the catheter at the proximal insertion end portion of the catheter tube, the hydration medium forming a residual coating on the inner surface of the catheter tube and contacting the hydrophilic coating on the transition of the at least one eyelet.

2. The urinary catheter product of claim 1, wherein the transition of the at least one eyelet comprises a rounded transition.

3. The urinary catheter product of claim 1, wherein the transition of the at least one eyelet comprises an angle or chamfer.

4. The urinary catheter product of claim 1, wherein the hydrophilic coating continuously extends from the outer surface of the catheter tube and onto the transition of the at least one eyelet.

5. The urinary catheter product of claim 1, wherein the hydrophilic coating continuously extends from the outer surface of the catheter tube, over the transition of the at least one eyelet and onto the inner surface of the catheter tube.

6. The urinary catheter product of claim 1, wherein the hydration medium of the residual coating is in contact with the hydrophilic coating and provides continuous hydration to the hydrophilic coating.

7. The urinary catheter product of claim 1, wherein the catheter tube includes a closed proximal terminal end, a section of the lumen of the catheter tube between the at least one eyelet and the closed proximal terminal end defines a reservoir containing an amount of residual hydration medium.

8. The urinary catheter product of claim 7, wherein the amount of residual hydration medium is in contact with the hydrophilic coating.

9. The urinary catheter product of claim 1, wherein the hydration medium is a foam comprising a liquid and gas.

10. The urinary catheter product of claim 1, wherein the hydration medium has a viscosity of from about 4 cP to about 7 cP at 25° C. and a shear rate of 5 s⁻¹.

11. (canceled)

12. The urinary catheter product of claim 1, wherein the hydration medium is pseudoplastic.

13. The urinary catheter product of claim 1, wherein the at least one eyelet comprises at least two eyelets.

14. The urinary catheter product of claim 1, wherein the hydration medium comprises water and a polyol.

15. The urinary catheter product of claim 14, wherein the polyol is glycerol.

16. The urinary catheter product of claim 1, wherein the hydration medium comprises a polysaccharide.

17. The urinary catheter product of claim 16, wherein the polysaccharide is xanthan gum.

18. The urinary catheter product of claim 1, wherein the hydration medium comprises a dispersant.

19. The urinary catheter product of claim 18, wherein the dispersant is sodium methyl cocyl taurate.

20. The urinary catheter product of claim 1, wherein the package comprises a sleeve.

21. (canceled)

22. The urinary catheter product of claim 1, wherein the hydration medium has a pH from about 3.6 to about 8 and an Osmolality of from about 40 mOsm/dm³ to about 590 mOsm/dm³.

23. (canceled)
